(19) United States
(12) Patent Application Publication
(10) Pub. No.: US 2012/0035743 A1
(43) Pub. Date: Feb. 9, 2012

(54) AMNION AND CHORION CONSTRUCTS AND USES THEREOF IN MINIMALLY INVASIVE SURGERIES

(75) Inventors: Robin R. YOUNG, Wayne, PA (US); Alessandro CANONACO, Caldwell, NJ (US); Richard M. JAY, Philadelphia, PA (US)

(73) Assignee: AFcell Medical, Parsippany, NJ (US)

(21) Appl. No.: 13/195,189

(22) Filed: Aug. 1, 2011

Related U.S. Application Data
(60) Provisional application No. 61/370,176, filed on Aug. 3, 2010.

(51) Int. Cl. A61F 2/02

(52) U.S. Cl. 623/23.72; 600/36

(57) ABSTRACT

A construct for use in a minimally invasive surgery is described. The construct contains an allograft having at least one layer of human amnion and chorion tissues, and is adapted for insertion into a small incision or a cannula employed in the minimally invasive surgery for access to the surgical site. The allograft has a shape appropriate for covering the surgical site. Methods of preparing the construct and using it in a minimally invasive surgery are also described. The products and methods improve the performance of the minimally invasive surgery, e.g., by reducing adhesions, scar formation while also reducing inflammation and risk of post-operative infection.

### CROSS-REFERENCE TO RELATED APPLICATIONS

This application is entitled to priority pursuant to 35 U.S.C. §119(e) to U.S. Provisional Patent Application No. 61/370,176, filed Aug. 3, 2010, which is hereby incorporated by reference herein in its entirety.

### BACKGROUND OF THE INVENTION

1. Field of Invention

Embodiments of the present invention relate to methods and products for improving minimally invasive surgery. In particular, embodiments of the present invention relate to constructs comprising an allograft having at least one layer of amnion and chorion tissues for use during minimally invasive surgeries. The constructs are adapted for the ease of insertion through a cannula for subsequent covering of the surgical site during a minimally invasive surgery.

2. Background of the Invention

Minimally invasive surgery (MIS) is an intervention done using incisions smaller than that used in general surgery and often employing a cannula for access to the surgical site and viewing technologies such as laparoscopes or arthroscopes to facilitate viewing the surgical site. A cannula is a tube through which surgeons can insert implants, instruments and such visualization devices as laparoscopes or arthroscopes. Minimally invasive surgery employs a camera, a light source and a cannula to visualize, remove tissue and implant therapeutic devices. During minimally invasive surgery several small portal incisions can be used instead of one large incision. The perceived benefit of minimally invasive surgery over an open procedure is that it will minimize the disruption of muscles and connective tissue and therefore improve the speed and completeness of a patient's post operative recovery. Minimally invasive surgeries have become very common, and are often performed as an outpatient procedure. There are various types of minimally invasive procedures, such as arthroscopy, to view joints and laparoscopy, to view the abdomen.

Having a small incision increases the difficulty of placing implants and instruments within the surgical area. The tension on the tissue can become high while the surgeon adjusts the instrumentation to get to the desired location. Instruments and implants have been designed for endoscopic surgery to minimize the difficulty of insertion and placement.

There has been some controversy on whether MIS procedures such as endoscopic surgery, reduce scarring and general recovery time. Regardless of the incision size, tissue is damaged during a surgical procedure. A product that effectively inhibits fibroblast formation, scarring and adhesion formation would be desirable as a wound covering or dressing for damaged tissue during MIS.

The amnion is a thin, cellular, extraembryonic membrane that forms the inner membrane of a closed placental sac surrounding and protecting an embryo in reptiles, birds, and mammals.

The sac contains the fetus and amniotic fluid or liquor amnii, in which the embryo is immersed, nourished and protected. Amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid.

The amnion has multiple functions, e.g., as a covering epithelium, as an active secretary epithelium, and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford et al., 1979, Am J Obstet Gynecol, 134:833-845. Amnioplastin, an isolated and chemically processed amniotic membrane, was used for continual dural repair, peripheral nerve injuries, conjunctival graft and flexor and muscle repair. See e.g., Chao et al., 1940, The British Medical Journal, March 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In recent years, there have been renewed interests in the application of amnion in ocular surface reconstruction, for example, as an allograph for repairing corneal defects. See, for example, Tsai and Tseng, Cornea. 1994 September;13(5):389-400; and Dua et al., Br. J. Ophthalmol 1999, 83:748-20 752. In addition, amnion and amniotic fluid have recently been used as sources of placental stem cells. See, e.g., U.S. Pat. No. 7,255,879 and WO 200073421.

Despite the clinical and published record regarding the safety and efficacy of amnion in broad surgical use, issues regarding reproducibility, safety and the precise form of amnion for each prospective indication have prevented amnion from achieving broad commercial distribution.

There is a need of improved methods and products for MIS that would effectively reduce inflammation and inhibit fibroblast formation, scarring and adhesion formation. The present invention relates to such improved methods and products.

### BRIEF SUMMARY OF THE INVENTION

In one general aspect, the present invention relates to a construct for use in a minimally invasive surgery. The construct comprises an allograft comprising at least one layer of human amnion and chorion tissues, wherein the construct is adapted for insertion into a small incision or a cannula employed in the minimally invasive surgery for access to the surgical site, and the allograft has a shape appropriate for covering the surgical site.

In another general aspect, the present invention relates to a method of preparing a construct for use in a minimally invasive surgery. The method comprises drying an allograft comprising at least one layer of human amnion and chorion tissues over a frame, preferably a rigid or semi rigid frame, of a shape appropriate for insertion into a small incision or a cannula employed in the minimally invasive surgery for access to the surgical site.

Another general aspect of the present invention relates to an improved minimally invasive surgery. The improvement comprises inserting a construct according to an embodiment of the present invention into a small incision or a cannula employed in the minimally invasive surgery for access to the surgical site to thereby cover the surgical site with the allograft.

Yet another general aspect of the present invention relates to a kit, which comprises:
(a) a construct for use in a minimally invasive surgery; and
(b) instructions on how to use the construct in the minimally invasive surgery,
wherein the construct comprises an allograft comprising at least one layer of human amnion and chorion tissues, the construct is adapted for insertion into a small incision or a cannula employed in the minimally invasive surgery for access to the surgical site, and the allograft has a shape appropriate for covering the surgical site.

In a preferred embodiment of the present invention, the human amnion and chorion tissues are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining amnion and chorion tissues from the placenta.

According to other embodiments of the present invention, the improvement to a minimally invasive surgery further comprises applying an amniotic fluid to the surgical site to thereby cover the surgical site with the amniotic fluid, and the kit further comprises an amniotic fluid and instructions on how to use the amniotic fluid in the minimally invasive surgery.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates the cross section of a collapsible construct having a plurality of spokes according to an embodiment of the present invention in a collapsed position;

FIG. 2 illustrates a configuration of a frame comprising a plurality of spokes that can be used in a construct according to an embodiment of the present invention;

FIGS. 3A and 3B illustrate constructs according to embodiments of the present invention in a collapsed position when inserted into a cannula during an MIS;

FIGS. 4A and 4B illustrate constructs as that illustrated in FIGS. 3A and 3B after the allografts exit the cannula in the MIS;

FIGS. 5A and 5B illustrate constructs according to embodiments of the present invention that are semi-spherical;

FIG. 6 illustrates a construct according to an embodiment of the present invention that is generally cylindrical with a C-shaped cross-section; and

FIG. 7 illustrates a construct according to an embodiment of the present invention that comprises an allograft dried over a cylindrical frame.

### DETAILED DESCRIPTION OF THE INVENTION

Unless defined otherwise, all technical and scientific terms used herein have the same meaning as commonly understood to one of ordinary skill in the art to which this invention pertains.

In this application, certain terms are used, which shall have the meanings as set in the specification. It must be noted that as used herein and in the appended claims, the singular forms “a,” “an,” and “the” include plural reference unless the context clearly dictates otherwise.

Embodiments of the present invention relate to an amnion and/or chorion construct for use in an MIS. The construct comprises an allograft comprising at least one layer of human amnion and chorion tissues. In a particular embodiment, the construct is collapsed, inserted into a patient through a cannula and then expanded in vivo at the surgical site during the MIS. The construct can be made by drying an allograft of amnion and/or chorion membranes into the required shape or over a frame, such as a resorbable frame, e.g., polymer mesh frame, or a disposable or stainless steel frame. The configuration of the construct allows for ease of insertion of the construct through a small incision or cannula and expansion of the allograft at the surgical site to thereby cover the site.

According to embodiments of the present invention, the constructs are made into shapes that can be inserted into a catheter or cannula for access to the surgical site during an MIS. Upon exiting the distal end of the catheter or cannula, the allograft of the construct expands in vivo to cover the surgical site. A frame, preferably a rigid or semi rigid resorbable polymer frame or stainless steel frame can be used in the construct to facilitate the insertion and subsequent expansion of the construct. In particular embodiments, the allograft used in constructs according to an embodiment of the present invention is in a collapsed state initially and then upon insertion into a patient expands to cover internal bone or tissue structures at the surgical site. A collapsible frame can be used in such constructs.

Embodiments of the present invention relate to several configurations of the amnion and/or chorion construct for use in an MIS procedure, which include, but are not limited to, endoscopic procedures.

In one embodiment of the present invention, the construct for use in an MIS is in a collapsed position.

In another embodiment of the present invention, the construct for use in an MIS further comprises a frame comprising a plurality of spokes that reinforce and facilitate the collapsing of the construct for ease of insertion through a small incision or cannula, and expanding of the allograft at the surgical site in the MIS. The spokes can be made of implantable resorbable rigid or semi rigid polymer mesh. One of such constructs in a collapsed position is illustrated in FIG. 1. In the construct, the plurality of spokes emanate from the center of the allograft and are secured to the patch along the lengths of the spokes. The spokes are movable between a collapsed position proximate each other and an expanded position extending radially from the center of the patch. The spokes are biased to the expanded position.

FIG. 2 illustrates a frame comprising a plurality of spokes that can be used in a construct according to an embodiment of the present invention. The polymer frame is configured with a central member with multiple spokes protruding out in a spiral direction. The multiple spokes are flexible with respect to the central member, and are positioned on an angle during drying to form a shape similar to that of a partially retracted umbrella. This configuration would allow for ease of insertion of the construct through a small incision or cannula and subsequent expansion at the surgical site.

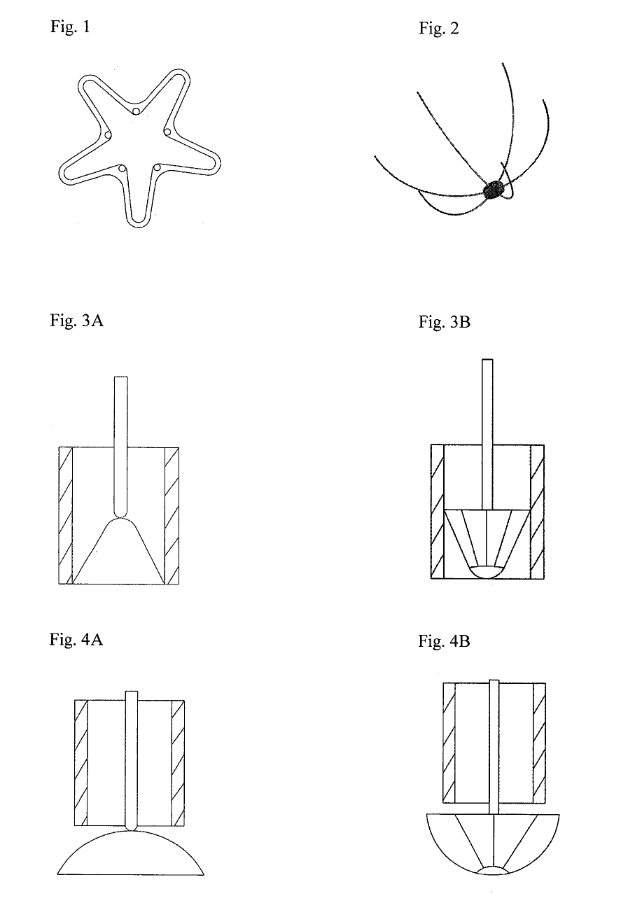

In an embodiment of the present invention, a central shaft is used to facilitate the insertion of the allograft into and through the cannula. FIGS. 3A and 3B illustrate constructs with a central shaft in a collapsed position inside a cannula. FIG. 4A and 4B illustrates such constructs after their allografts exiting the cannula. The construct illustrated in FIG. 3B and 4B has a plurality of spokes.

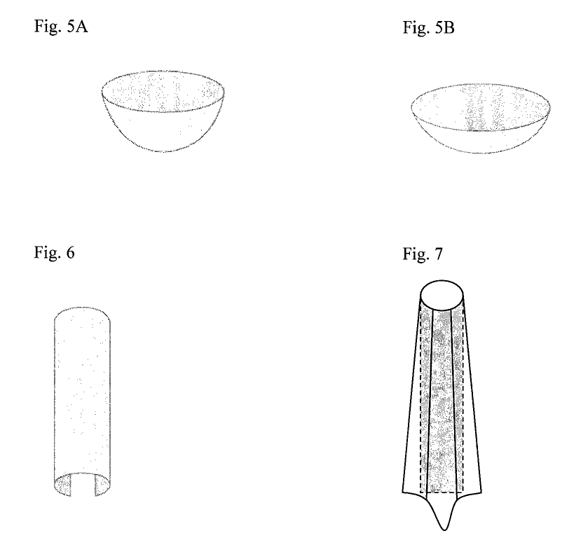

In one embodiment of the present invention, the construct for use in an MIS is semi-spherical to allow for ease of insertion through a small incision or cannula. See FIGS. 5A and 5B.

In yet another embodiment of the present invention, the construct as that illustrated in FIGS. 5A and 5B further comprise a rigid or semi rigid frame of the semi-spherical shape for ease of insertion through a small incision or cannula.

In one embodiment of the present invention, the construct for use in an MIS is generally cylindrical with a C-shaped cross-section to allow for ease of insertion through a small incision or cannula. See FIG. 6.

In another embodiment of the present invention, the construct as that illustrated in FIG. 6 further comprises a rigid or semi rigid frame of the generally cylindrical shape with a C-shaped cross-section for ease insertion through a small incision or cannula.

In yet another embodiment of the present invention, the construct as that illustrated in FIG. 6 further comprises a rigid or semi rigid frame that is collapsible.

In yet another embodiment of the present invention, the construct for use in an MIS comprises a cylindrical frame. See FIG. 7. An allograft comprising at least one layer of human amnion and chorion tissues of a rectangular or circular sheet is dried over the cylindrical frame to allow for ease insertion through a small incision or cannula.

In one embodiment of the present invention, one or more corners of the construct or allograft are rounded or flatted to prevent the corners from catching during implantation. In view of the present disclosure, any method known to those skilled in the art can be used to make the corners of the construct or allograft round or flatten.

In one embodiment of the present invention, the allograft in the construct can carry one or more therapeutic agents, such as morphogenic proteins, small molecule compounds, pharmaceutical agents, anti-microbial agents, anti-inflammatory agent, agents that prevent scarring, adhesions and tethering of internal tissue at or near the surgery site, analgesics, etc., to further improve the performance and reduce the complications of MIS. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

In another general aspect, embodiments of the present invention relate to a method of preparing a construct for use in an MIS. The method comprises drying an allograft comprising at least one layer of human amnion and chorion tissues over a frame, preferably a rigid or semi rigid frame of a shape appropriate for insertion into a small incision or a cannula employed in the minimally invasive surgery for access to the surgical site. The frame can be any of the shapes described above, e.g., semi-spherical, cylindrical, or generally cylindrical with a C-shaped cross-section. The frame can also comprise a plurality of spokes as described above.

In an embodiment of the present invention, when a disposable frame is used, the dried tissue retains the shape of the frame when removed from the frame or could be packaged and sterilized with a disposable frame to retain its shape prior to use. The disposable frame can be removed and discarded prior to the use of the tissue. The disposable frame can be longer than the tissue for ease of handling and removal.

This implantable and resorbable frame could be a mesh or a solid frame with several holes throughout.

The allograft, such as that comprising one or more layers of human amnion and/or chorion tissues, is bonded to the frame by various methods in view of the present disclosure, such as, drying the tissue on the frame, using a resorbable adhesive, keeping the tissue wet and laying it on the frame, or freezing the tissue on the frame.

Another general aspect of the present invention relates to an improved method of performing an MIS. The improvement comprises inserting a construct according to embodiments of the present invention into the small incision or the cannula employed in the MIS to thereby cover the surgical site with the allograft.

The improvement can be applied to any procedure of MIS in view of the present disclosure. The circumference of the allograft can be slightly greater than half a full circle to allow ease of insertion. The circumference of the allograft can be larger than the surgical site it will be implanted on so that when hydrated it will fully encase the surgical site.

In another embodiment of the present invention, a construct comprising at least one layer of amnion and chorion tissues is used to cover a skin incision resulting from the MIS. The allograft patch can be of any size suitable for covering the sutures or other type of tissue injuries at skin incision.

Preferably, a relatively thick layer of allograft is used to cover the skin incision. In one embodiment of the invention, the allograft patch has a thickness of about 2 mm to 4 mm. It can have multiple layers of amnion or a combination of multiple layers of amnion and chorion in any combination of amnion and chorion.

In another embodiment of the present invention, amniotic fluid can be applied into the small incision or the cannula employed in the MIS to thereby cover the surgical site with the amniotic fluid. The amniotic fluid can also be applied to cover a skin incision resulting from the MIS.

The amniotic fluid and the construct can be applied individually or in combination during the surgery. Preferably, the amniotic fluid is processed so that it has a relatively high viscosity for ease of application and for remaining in the desired area after the application. Methods known to those skilled in the art can be used to prepare amniotic fluid with a relatively high viscosity in view of the present disclosure.

The present invention overcomes shortcomings of the prior art by making human allograft membranes usable as surgical implants in an MIS.

There are several attributes which make an allograft having at least one of amnion and chorion tissues a preferred material for use in an MIS. Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J Ophthalmol. 85(4):444-9) and produces natural inhibitors of matrix metalloproteases (MMPs) expressed by infiltrating polymorphonuclear cells and macrophages. Hao et al., 2000, Cornea,19(3):348-52; Kim et al., 2000, Exp Eye Res. 70(3):329-37). Amnion also down-regulates TGF-13 and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it a potential allograft candidate to be used in an MIS.

Human allograft amnion and chorion have the ability to prevent scarring, reduce inflammation, inhibit microbial infection and improve healing. During an MIS, the surgeon is required to work in very tight spaces. Covering the surgical site with a flat sheet of membrane that improves healing can be extremely difficult for the surgeon. By creating and using a collapsible and expandable construct, allografts of human anmion and/or chorion can now be delivered through the cannula to allow subsequently covering of the surgical site with ease. The allografts have the ability to reduce adhesions, scar formation while also reducing inflammation and risk of post-operative infection.

Amnion, chorion and amniotic fluid used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The present invention encompasses a kit comprising a construct for use in an MIS and instructions on how to use the construct in the MIS. Any of the constructs for use in an MIS according to embodiments of the present invention can be included in the kit. The construct comprises an allograft comprising at least one layer of human amnion and chorion tissues. The construct is adapted for insertion into a small incision or a cannula employed in the minimally invasive surgery for access to the surgical site, and the allograft has a shape appropriate for covering the surgical site. In a preferred embodiment, the kit comprises a plurality of constructs for MIS, and at least two of the plurality of constructs have different shapes or sizes suitable for covering different surgical sites. The allograft in the construct can further comprise one or more therapeutically active agents, such as anti-microbial agents, growth enhancing agents, anti-inflammatory agents, analgesics, etc.

According to an embodiment of the present application, the kit further comprises an amniotic fluid and instructions on how to use the amniotic fluid in the minimally invasive surgery.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A construct for use in a minimally invasive surgery, the construct comprising an allograft comprising at least one layer of human amnion and chorion tissues, wherein the construct is adapted for insertion into a small incision or a cannula employed in the minimally invasive surgery for access to the surgical site, and the allograft has a shape appropriate for covering the surgical site.

2. The construct of claim 1, further comprising a rigid or semi rigid frame of a shape appropriate for insertion through the small incision or the cannula, the frame being disposable or implantable and resorbable.

3. The construct of claim 2, wherein the frame comprises a plurality of spokes emanating from the center of the allograft and secured to the patch along the lengths of the spokes, the spokes being movable between a collapsed position proximate each other and an expanded position extending radially from the center of the patch, and the spokes being biased to the expanded position.

4. The construct of claim 2, wherein the frame is collapsible.

5. The construct of claim 2, wherein the frame is semi-spherical, cylindrical, or generally cylindrical with a C-shaped cross-section.

6. The construct of claim 1, further comprising a central shaft, the allograft being movable between a collapsed position enwrapping the central shaft along its longitudinal axis and an expanded position contacting only one end of the central shaft at the center of the patch, wherein the central shaft is disposable or implantable and resorbable.

7. The construct of claim 1, having one or more rounded or flattened corners.

8. The construct of claim 1, further comprising one or more therapeutic agents to further improve the performance and reduce the complications of the minimally invasive surgery.

9. A method of preparing a construct for use in a minimally invasive surgery, the method comprising drying an allograft comprising at least one layer of human amnion and chorion tissues over a frame of a shape appropriate for insertion into a small incision or a cannula employed in the minimally invasive surgery for access to the surgical site.

10. The method of claim 9, wherein the frame comprises a plurality of spokes, upon drying, the spokes emanating from the center of the allograft and secured to the patch along the lengths of the spokes, the spokes being movable between a collapsed position proximate each other and an expanded position extending radially from the center of the patch, and the spokes being biased to the expanded position.

11. The method of claim 9, wherein the frame is collapsible.

12. The method of claim 9, wherein the frame is semi-spherical, cylindrical, or generally cylindrical with a C-shaped cross-section.

13. The method of claim 9, further comprising placing a central shaft at the center of the patch, upon drying, the allograft being movable between a collapsed position enwrapping the central shaft along its longitudinal axis and an expanded position contacting only one end of the central shaft at the center of the patch, and the central shaft being disposable or implantable and resorbable.

14. The method of claim 9, wherein the human amnion and chorion tissues are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining amnion and chorion tissues from the placenta.

15. In a method of performing a minimally invasive surgery, the improvement comprising inserting a construct of claim 1 into the small incision or the cannula employed in the minimally invasive surgery to thereby cover the surgical site with the allograft.

16. In the method of claim 15, the improvement further comprising applying one or more allografts comprising at least one layer of human amnion and chorion tissues over one or more suture lines and incisions resulting from the minimally invasive surgery to form a cover and barrier over the suture lines and the incisions.

17. In the method of claim 15, wherein the construct further comprises a rigid or semi rigid frame of a shape appropriate for insertion through the small incision or the cannula, the frame being disposable or implantable and resorbable.

18. In the method of claim 15, the improvement further comprising applying an amniotic fluid to the surgical site to thereby cover the surgical site with the amniotic fluid.

19. A kit comprising
(a) a construct for use in a minimally invasive surgery; and
(b) instructions on how to use the construct in the minimally invasive surgery,
wherein the construct comprises an allograft comprising at least one layer of human amnion and chorion tissues, the construct is adapted for insertion into a small incision or a cannula employed in the minimally invasive surgery for access to the surgical site, and the allograft has a shape appropriate for covering the surgical site.

20. The kit of claim 19, wherein the frame comprises a plurality of spokes emanating from the center of the allograft and secured to the patch along the lengths of the spokes, the spokes being movable between a collapsed position proximate each other and an expanded position extending radially from the center of the patch, and the spokes being biased to the expanded position.
